(19) United States
(12) Patent Application Publication
(10) Pub. No.: US 2023/0052186 A1
(43) Pub. Date: Feb. 16, 2023

(54) FOREIGN BODY RETRIEVAL DEVICE

(71) Applicant: The University of Chicago, Chicago, IL (US)

(72) Inventors: Seon-Kyu LEE, Chicago, IL (US); Thuong Van HA, Chicago, IL (US)

(73) Assignee: The University of Chicago, Chicago, IL (US)

(21) Appl. No.: 17/758,896

(22) PCT Filed: Jan. 12, 2021
(86) PCT No.: PCT/US2021/070022

§ 371 Date: Jul. 15, 2022

Related U.S. Application Data
(60) Provisional application No. 62/961,417, filed on Jan. 15, 2020.

(51) Int. Cl. A61B 17/221; A61F 2/01

(52) U.S. Cl. CPC A61B 17/221; A61F 2/011; A61B 2017/22045

(57) ABSTRACT

The present disclosure includes a device for retrieving foreign material within a blood vessel of a patient. Some foreign retrieval devices include an elongated body configured to be disposed within a catheter, the elongated body having a proximal end, and a distal end opposite the proximal end; and one or more arms, each arm having a proximal end coupled to the distal end of the elongated body, and a distal end extending toward the proximal end of the elongated body. In some foreign retrieval devices, each of the arms is configured to be actuated from a closed position to an open position.

### CROSS-REFERENCE TO RELATED APPLICATIONS

This application claims the benefit of priority of U.S. Provisional Patent Application No. 62/961,417 filed Jan. 15, 2020, which is hereby incorporated by reference in its entirety.

### FIELD OF INVENTION

The present invention relates generally to apparatuses and methods for retrieval of foreign objects from a blood vessel, and more specifically, but not by way of limitation, removal of an Inferior Vena Cava filter under fluoroscopy control.

### BACKGROUND

An Inferior Vena Cava (“IVC”) filter is a small device that is placed in your inferior vena cava to prevent blood clots from moving through your blood into your lungs, brain, heart or other major organ. While IVC filters help to prevent a heart attack or stroke, they are prone to create complications when placed within a blood vessel of a patient for a prolonged period of time. For example, some IVC filters have been found to break apart in the blood stream and damage the vein. IVC Filters can also increase the risk of caval thrombosis or narrowing, IVC or adjacent vessel/organ perforation, extension of lower extremity deep vein thrombosis, filter fracture, filter migration, embolization, and more. Until recently, IVC filters were available only as permanently implanted devices. Newer filters, called optionally retrievable filters, may be permanently implanted or have the option to potentially be removed from the blood vessel once the risks associated with the blood clots are eliminated or reduced.

Most optionally retrievable filters have a small hook at one end. Typically, a snare is inserted into the blood vessel and used to latch onto the small hook, and once attached, to withdraw the filter. In many cases, retrieval of the IVC filter using the hook and snare techniques is difficult and time-consuming. Consequently, IVC filter removal using the hook and snare technique is often unsuccessful. In cases where snare retrieval is unsuccessful, an advanced complex filter removal technique is likely required to remove the IVC filter. These complex techniques can require multiple access points, higher risks of complications, as well as other potential complications.

Further, this method of IVC filter removal is only available if the IVC filter design includes a hook and is not feasible for removing older or alternatively designed IVC filters. Currently, IVC filter retrieval is further complicated by the plethora of IVC filter designs currently available on the market. If an IVC filter design is changed or modified, then the removal method may be rendered obsolete. Thus, there exists a need for an easy to use IVC filter retrieval device that is operable with several types of IVC filters.

### SUMMARY

The present disclosure describes devices, systems, and methods for retrieval of foreign material within a blood vessel of a patient. Notably, the device is able to successfully retrieve IVC filters regardless of the design of the filter. Some devices and methods of the present disclosure may be used for retrieving an object within a blood vessel of a patient. In some configurations, the device includes an elongated body configured to be disposed within a catheter and one or more arms configured to be actuated between a closed position and an open position for engaging the foreign material between the arms and the elongated body. The elongated body may include a proximal end, and a distal end opposite the proximal end and. In some configurations, each arm includes a proximal end coupled to the distal end of the elongated body and a distal end extending toward the proximal end of the elongated body in the closed position, the distal end being free. In the open position, the arm extends radially away from the elongated body to define a first maximum transverse dimension of the device and in the closed position, the distal end of the arm is closer to the elongated body than when in the open position to define a second maximum transverse dimension of the device that is smaller than the first maximum transverse dimension.

In some configurations, the devices includes a collar disposed around the elongated body and one or more support members, each support member coupled to the collar and a respective arm of the one or more arms. In some such configurations, each support member is configured such that relative movement of the collar relative to the elongated body causes the respective arm to move between the open position and the closed position. In some configurations, each support member comprises a first end coupled to the elongated body and a second end coupled to the respective arm. The first end of each support member may be pivotably coupled to the collar such that as the collar moves toward the distal end of the elongated body, the distal end of each of the arms move radially outward away from the elongated body.

In some configurations, the device includes an inner catheter to which the collar is coupled. The elongated body may define a lumen extending through the elongated body between proximal end and distal end. The proximal end of each arm may be pivotably coupled to the elongated body and the second end of each support member may be pivotably coupled to the respective arm. In some configurations, the elongated body is movable relative to the collar such that when a distance between the collar and the distal end of the elongated body increases, the distal end of each arm moves towards the proximal end of the elongated body. In some configurations, the elongated body is movable relative to the collar such that when the distance between the collar and the distal end of the elongated body increases, the distal end of each arm moves radially towards the elongated body. In some configurations, the elongated body is movable relative to the collar such that when the distance between the collar and the distal end of the elongated body decreases, the distal end of each arm moves radially away from the elongated body.

In some configurations, the first transverse dimension is greater than or equal to 2.667 mm and/or the second transverse dimension is less than or equal to 2.33 mm. Additionally, or alternatively, each of the arms has a maximum transverse dimension that is less than or equal to 0.35 in. In some configurations, an inner diameter of the outer catheter is greater than or equal to the second maximum transverse dimension.

Some systems of the present disclosure include a kit. In some systems, the kit includes a package where the device is sterile and sealed within the package.

Some of the present methods include advancing a retrieval device, having an elongated body and one or more arms each having a proximal end coupled to the elongated body, though the blood vessel toward and past at least a portion of an object disposed within the blood vessel, actuating the one or more arms such that the one or more arms expand radially away from the elongated body, retracting the retrieval device in the blood vessel toward the object to engage the object between the one or more arms and the elongated body; and retracting the retrieval device with the object engaged thereto through the blood vessel.

In some methods, engaging the object between the one or more arms and the elongated body comprises actuating the one or more arms such that the distal ends of the one or more arms move radially towards the elongated body to engage the object between the one or more arms and the elongated body. The retrieval device is may be advanced through a catheter disposed within the blood vessel. In some methods, the retrieval device is advanced to extend out of a distal end of the catheter to engage the object in the blood vessel. In some methods, the retrieval device is retracted through the catheter disposed within the blood vessel after engaging the object in the blood vessel. Additionally, or alternatively, a guidewire may be used to advance and retract the retrieval device through the blood vessel.

The term “coupled” is defined as connected, although not necessarily directly, and not necessarily mechanically; two items that are “coupled” may be unitary with each other. The terms “a” and “an” are defined as one or more unless this disclosure explicitly requires otherwise. The term “substantially” is defined as largely but not necessarily wholly what is specified (and includes what is specified; e.g., substantially 90 degrees includes 90 degrees and substantially parallel includes parallel), as understood by a person of ordinary skill in the art. In any disclosed configuration, the term “substantially” may be substituted with “within [a percentage] of” what is specified, where the percentage includes 0.1, 1, 5, and 10 percent.

Further, an apparatus or system that is configured in a certain way is configured in at least that way, but it can also be configured in other ways than those specifically described.

The terms “comprise” (and any form of comprise, such as “comprises” and “comprising”), “have” (and any form of have, such as “has” and “having”), and “include” (and any form of include, such as “includes” and “including”) are open-ended linking verbs. As a result, an apparatus that “comprises,” “has,” or “includes” one or more elements possesses those one or more elements, but is not limited to possessing only those elements. Likewise, a method that “comprises,” “has,” or “includes” one or more steps possesses those one or more steps, but is not limited to possessing only those one or more steps.

Any configuration of any of the apparatuses, systems, and methods can consist of or consist essentially of—rather than comprise/include/have—any of the described steps, elements, and/or features. Thus, in any of the claims, the term “consisting of” or “consisting essentially of” can be substituted for any of the open-ended linking verbs recited above, in order to change the scope of a given claim from what it would otherwise be using the open-ended linking verb.

The feature or features of one configuration may be applied to other configurations, even though not described or illustrated, unless expressly prohibited by this disclosure or the nature of the configurations.

Some details associated with the configurations described above and others are described below.

### BRIEF DESCRIPTION OF THE DRAWINGS

The following drawings illustrate by way of example and not limitation. For the sake of brevity and clarity, every feature of a given structure is not always labeled in every figure in which that structure appears. Identical reference numbers do not necessarily indicate an identical structure. Rather, the same reference number may be used to indicate a similar feature or a feature with similar functionality, as may non-identical reference numbers. The figures are drawn to scale (unless otherwise noted), meaning the sizes of the depicted elements are accurate relative to each other for at least the configuration depicted in the figures.

FIG. 1 is a schematic perspective view of a configuration of the present filter retrieval device.

FIGS. 2A-2B are side views of some of the components of filter retrieval device of FIG. 1 in an open position and a closed position, respectively.

FIGS. 3A-3B are top views of a distal end of a grapple of the present filter retrieval device in the open and closed positions, respectively.

FIGS. 4A-4B are schematic side views of an arm and a support of the grapple moving from the open and closed position, respectively.

FIG. 5 is s flowchart illustrating an example of one method of engaging an object using the filter retrieval device.

FIGS. 6A-6E are schematic views of a configuration of the filter retrieval device shown in use retrieving a filter from an inferior vena cava of a patient

### DETAILED DESCRIPTION

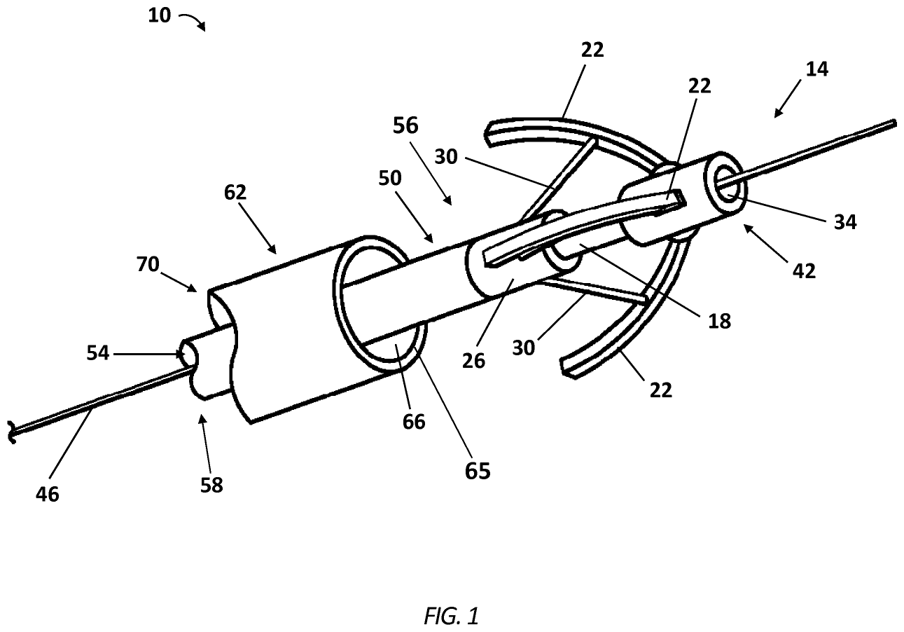

Referring now to the drawings, and more particularly to FIG. 1, shown therein and designated by the reference numeral 10 is an embodiment of a foreign body retrieval device configured to retrieve material within a blood vessel of a patient. For example, foreign body retrieval device 10 could be a filter retrieval device for removal of an inferior vena cava (“IVC”) filter that has been previously placed in a patient. Although the present description is provided with respect to a filter retrieval device, it is contemplated that the description applies generally to foreign body retrieval devices for retrieval of objects from hard to access confined spaces. For example, foreign body retrieval device may be configured for use as an endovascular device, endobronchial device, or the like.

Referring to FIG. 1, filter retrieval device 10 comprises a grapple 14 and one or more catheters (e.g., 50, 62). In some configurations, grapple 14 may comprise an elongated body 18, one or more arms 22, a collar 26, and one or more supports 30. As shown in FIG. 1, elongated body 18 may define a lumen 34 extending between a proximal end 38 and a distal end 42 of the elongated body, the distal end 42 being opposite of the proximal end 38. In some configurations, a guidewire 46 may be disposed within lumen 34 to assist in navigating elongated body 18 through a blood vessel.

In the depicted configurations, filter retrieval device 10 comprises an inner catheter 50 that defines a lumen 54 extending between a distal end 56 and a proximal end 58 of the inner catheter. In the configurations shown, collar 26 is coupled to distal end 56 of inner catheter 50. In other configurations, collar 26 may be unitary with inner catheter 50. In the depicted configurations, elongated body 18 is at least partially disposed within lumen 54 of inner catheter 50, while arm(s) 22, support(s) 30, and collar 26 are disposed outside of lumen 54 of the inner catheter 50. In some embodiments, an outer catheter 62 may be used to guide inner catheter 50 and grapple 14 within the blood vessel. In these configurations, lumen 66 of outer catheter 62 may have a diameter greater than that of inner catheter 50 to allow removal of the grapple 14, inner catheter 50, and the foreign material through the lumen of outer catheter. In this way, risk of damage to the blood vessel by the component or foreign body may be minimized. In such configurations, elongated body 18 may be flexible to be easily guided through outer catheter 62. Similarly, inner catheter 50 and/or outer catheter 62 may be flexible to easily navigate through the blood vessels of a patient. In this manner, elongated body 18, inner catheter 50, and outer catheter 62 can be manipulated by an operator to navigate grapple 14 to a foreign object (e.g., 108) and to retract the grapple along with the foreign object as will be described below.

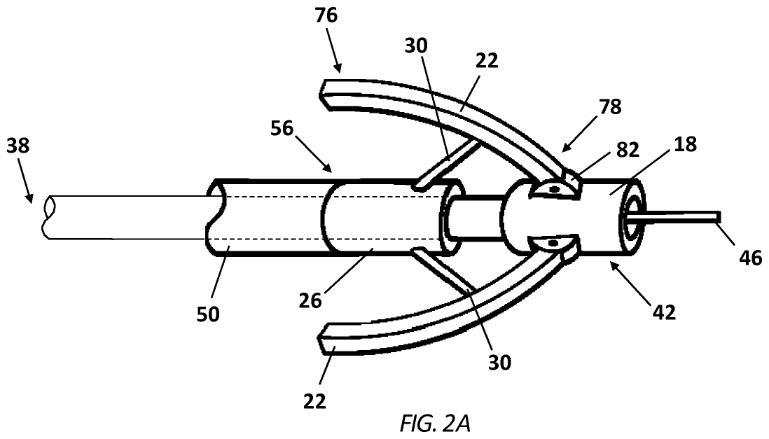

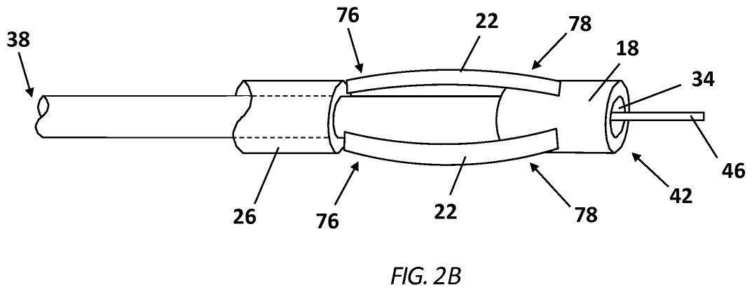

In the configurations shown in FIGS. 2A-2B, a side view of grapple 14 and inner catheter 50 is shown. For example, FIG. 2A in an example of foreign body retrieval device 10 in a first position (e.g., open position), and FIG. 2B is an example of foreign body retrieval device 10 in a second position (e.g., closed position). As described in further detail herein, foreign body retrieval device 10 is movable between the first and second positions to engage, and remove, a foreign object from hard to access spaces (e.g., a blood vessel). Notably, the arm(s) 22 of grapple 14 extend backwards (i.e., toward proximal end) from distal end 42 of elongated body 18. In this way, grapple 14 may act as a reverse prong device having arm(s) 22 that hook the foreign object (e.g., 108).

As shown, arm(s) 22 may pivot as foreign body retrieval device moves between the first and second positions. In the depicted configuration, arm(s) 22 include a distal end 76 and a proximal end 78 that is coupled to elongated body 18. For example, proximal end 78 of arm(s) may be coupled to distal end 42 of elongated body such that distal end 76 of arm(s) extends toward proximal end 38 of elongated body. In the depicted configuration, proximal end 78 of each arm is coupled to elongated body 18 via a hinge 82. In other configurations, each arm 22 may be fixedly, slidably, or pivotably coupled to elongated body 18 in any suitable fashion, such as, for example, via fasteners, adhesives, and/or the like. For example, in configurations where arm(s) 22 are fixedly coupled to body 18, the arm(s) 22 may comprise a flexible material to allow each arm to bend between the first and second positions while remaining fixed to the elongated body 18. The distal end 76 of each arm is free in order to engage the foreign object for removal. The foreign object can slide along the arm 22 from the distal end towards the proximal end 78.

In the depicted configuration, collar 26 is disposed around, or coupled to, the elongated body 18 near the distal end 42 and is moveable relative to the elongated body 18. In the depicted configuration, a support member 30 (e.g., support) connects each arm 22 to collar 26 or inner catheter 50 and helps to actuate the arm 22 between open and close positions as the collar 26 moves relative to the elongated body 18. In this way, each component (e.g., support(s) 30, arm(s) 22, elongated body 18, and collar 26) of grapple 14 is interconnected to allow movement of the grapple 14 in a variety of different ways. In the depicted configurations, each support 30 is coupled to a respective single arm (e.g., 22) of the one or more arms(s); it is however contemplated that in other configurations, each support 30 could be coupled to more than one arm 22 and/or each arm 22 may be coupled to more than one support 30.

In the depicted configuration, a first end 98 of each support 30 is coupled to collar 26 and a second end 100 of each support is coupled to the respective arm (e.g., 22a). The second end 100 of each support 30 is connected to the respective arm 22 at a location between the proximal end 78 and the distal end 76. The distal end 76 is thus free, and each arm 22 has a free portion extending from the distal end 76 to the connection with the second end 100 of the support 30 by which the arm 22 can engage the foreign object such as an IVC filter. In particular, the free portion of the arm 22 can be manipulated appropriately with respect to the foreign object to engage and secure the foreign object between the arm 22 and the elongated body 18 or collar 26.

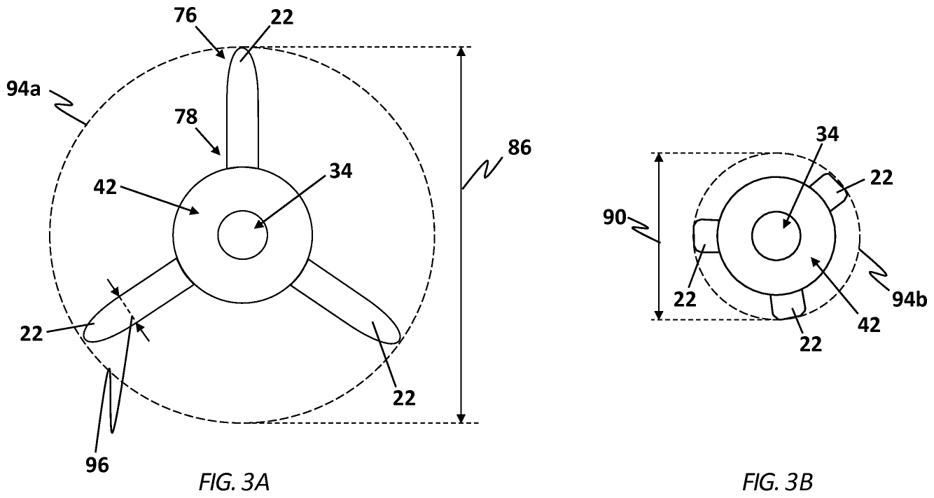

As shown in FIGS. 2A and 2B, elongated body 18 is moveable relative to inner catheter 50 and/or collar 26. In the depicted configurations, elongated body 18 may be moved relative to inner catheter 50 such that each arm 22 is actuated from a closed position (e.g., FIG. 2A) to an open position (e.g., FIG. 2B). As shown in FIG. 2A, when arm(s) 22 are in the first position (e.g., open position), the distal end 76 of each arm 22 extends radially away from elongated body 18 to define a first maximum transverse dimension 86 (FIG. 3A) of grapple 14. As shown in FIG. 2B, when arm(s) 22 are in the second position (e.g., closed position), distal end 76 of each arm (e.g., 22) moves closer to the elongated body 18 than when in the open position to define a second maximum transverse dimension 90 (FIG. 3B). In the depicted configurations, first maximum transverse dimension 86 (i.e., when grapple 14 is in the open position) is greater than second maximum transverse dimension 90 and both maximum transverse dimensions 86, 90 are less than the dimension of the blood vessel or other cavity from which the foreign material is being removed such that the grapple 14 may be operated in the open and closed positions to retrieve foreign material from the blood vessel or other cavity. When the arms 22 are in an open position, the grapple 14 could be moved towards a foreign object 108, such as an IVC filter, to engage the foreign object with the distal end 76. The arms 22 may then be moved from the open position to the closed position to secure the IVC filter (e.g., 108) for removal from the blood vessel. Specifically, grapple 14 may be moved in the blood vessel past a foreign object (e.g., 108), and the arm(s) 22 could be moved to the open position, after which the grapple is pulled back towards the object to engage (e.g., hook) the object with the distal end 76. In some such configurations, arms(s) 22 may be moved to the closed position after the foreign object has been engaged to secure (e.g., clamp) the object to foreign body retrieval device 10. In this way, grapple 14 may utilize arm(s) 22 to both hook and clamp the foreign object while retrieving the object.

Referring to FIGS. 3A and 3B, grapple 14 can be shaped and sized to fit within a blood vessel (e.g., inferior vena cava) of a patient in both the open position and the closed position. When grapple 14 is, for example, in the open position, the maximum transverse dimension (e.g., 86) of the grapple can be greater than or equal to any one of, or between any two of: 1.0, 1.33, 1.67, 2.0, 2.33, 2.67, 3.0, 3.33, 3.67, 4.0, 4.33, 4.67, 5.0, 5.33, 5.67, 6.0, 6.50, 7.0, 7.50, 8.0, 8.50, 9.0, 9.50, 10.0, 10.50, 11.0, 12.0, 13.0, 14.0, 15.0, 17.50, 20.0, 25.0, 28.0 or 30 millimeters (mm.) (e.g., between 2.67 and 10.47 mm., such as approximately 3.33 mm.). Likewise, when grapple 14 is, for example, in the closed position, the maximum transverse dimension (e.g., 90) of the grapple can be greater than or equal to any one of, or between any two of: 0.33, 0.67, 1.0, 1.33, 1.67, 2.0, 2.33, 2.67, 3.0, 3.33, 3.67, 4.0, 4.33, 4.67, 5.0, 5.33, 5.67, 6.0, 6.50, 7.0, 7.50, 8.0, 8.50, 9.0, 9.50, 10.0, 10.50, 11.0, 12.0, 13.0, 14.0, 15.0, 18.0, or 20 millimeters (mm.) (e.g., between 1.33 and 6.28 mm., such as approximately 2.33 mm). In this way, grapple 14 may be able to navigate through the blood vessel when in either the open position or the closed position.

Grapple 14 may define a circumference (94a, 94b) (FIG. 3A and 3B) that circumscribes arms 22 that can be equal to any one of, or between any two of: 3, 4, 5, 6, 7, 8, 9, 10, 11, 12, 13, 14, 15, 16, 17, 18, 19, 20, 22, 24, 26, or 28, 30, 32, 34 French gauge (Fr). Circumference (e.g., 94a, 94b) of grapple 14 is larger in the open position (e.g., 94a) than in the closed position (e.g., 94b). In some configurations, lumen 66 of outer catheter 62 may be sized to be greater than circumference 94a of grapple 14 such that the grapple may be disposed within the lumen when the grapple is in the open position. In some configurations, lumen 54 of inner catheter 50 may be sized to surround grapple 14 when in the closed position. For example, lumen 54 may have a diameter that is greater than the second maximum transverse dimension 90 of grapple 14. However, in other embodiments, lumen 54 may comprise a diameter than is greater than or equal to first maximum transverse dimension 86 of grapple 14. In yet other configurations, lumen 54 may surround only elongated body 18.

In the depicted configuration, each arm 22 is sized and shaped to facilitated engagement of a foreign material in the blood vessel. For example, grapple 14 and arms 22 may be optimized based on a size of the blood vessel. In uses with larger vessels, such as iliac veins, IVC or aorta, a stronger and more semi-rigid material (e.g., stainless steel) may be used. On the other hand, in uses with smaller, more tortuous vessels, such as intracranial or extremity vessels, a more flexible material (e.g., nitinol) may be used. In either configuration, the grapple is sized to move within the vessel without damaging the vessel and a portion of the elongated body 18 may be encased in a plastic type sheath for additional protection.

As shown, grapple 14 comprises three arms 22; however in other configurations, grapple may comprise one, two, four or more arms (e.g., 22). In some configurations, arm(s) 22 have a uniform thickness, while in other configurations, the arm(s) may be tapered at distal end 76. In the depicted configuration, three arms 22 are arranged circumferentially around elongated body 18 and spaced at regular intervals around a circumference of the elongated body 18. In other configurations, the arm(s) 22 may be spaced at irregular intervals around elongated body 18, or arranged in any other suitable manner that would allow the arm(s) 22 to engage an object. In the configuration shown, each arm 22 is positioned around elongated body at equiangular intervals. Arm(s) 22 are sized to fit within a blood vessel of a patient. For example, each arm 22 may have a maximum transverse dimension 96 (e.g., thickness) that is greater than or equal to any one of, or between any two of: 0.05, 0.1, 0.15, 0.2, 0.25, 0.3, 0.35, 0.4, 0.45, 0.5, 0.55, 0.6, 0.65, 0.7, 0.75, 0.8, 0.9, 1.0 inches (in.) (e.g., approximately 0.35 in).

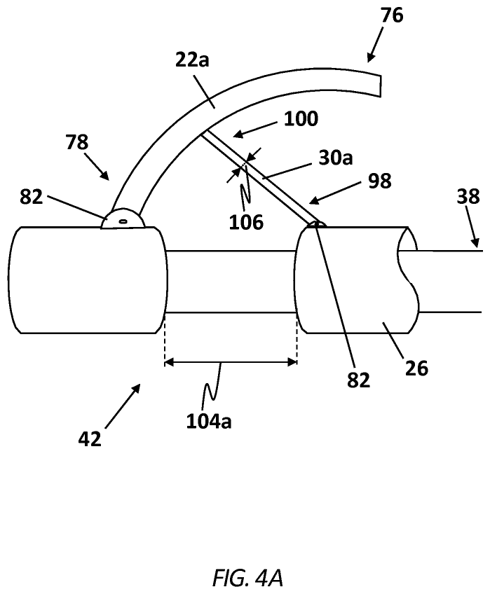

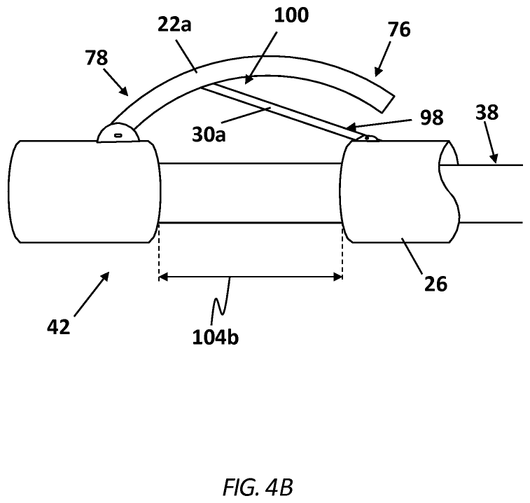

As shown, arm(s) 22, support(s) 30, and collar 26 operate together to move grapple 14 from the open position to the closed position, or vice versa. Referring to FIGS. 4A and 4B the movement of a single arm (e.g., 22a) and a respective support member (e.g., 30a) as grapple 14 transitions from the open position (e.g., FIG. 4A) to the closed position (e.g., FIG. 4B) will be described. As shown, collar 26 is disposed around at least a portion of elongated body 18. In this configuration, elongated body 18 is movable relative to collar 26 such that a distance 104 between the collar 26 and distal end 42 of the elongated body 18 can be increased or decreased to move between the open position and the closed position. As distance 104 is increased, distal end 76 of each arm moves radially towards elongated body 18 (e.g., toward to the closed position). In the depicted configuration, as distance 104 increases (e.g., moving from 104a to 104b) arm(s) 22 move toward the closed position (e.g., FIGS. 2B and 3B) and distal end 76 of each arm 22 moves towards proximal end (e.g., 38) of elongated body 18. If distance 104 is decreased, elongated body 18 moves away from collar 26 and distal end 76 of each arm moves radially away from the elongated body 18 and towards distal end 42 of the elongated body. In this way, grapple 14 is selectively movable to any position between the open position and the closed position. The depicted embodiments may be employed to retrieve a wide range of foreign material within the blood vessel or other body cavity. Specifically, filter retrieval device 10 may remove multiple types of IVC filters even if the IVC filter is not designed to be removed (e.g., does not comprise a hook or other retrieving mechanism). The backward-prong like structure of the arms enables the grapple to engage and secure different parts of the IVC filter other than a hook. For example, grapple 14 may be positioned past a portion of the IVC filter and drawn backwards towards the portion of the IVC filter so that arm(s) 22 engage the portion of the IVC filter. In this way, arm(s) 22 may be able to hook and/or clamp onto the portion of the IVC filter to engage the IVC filter.

In the depicted configurations, first end 98 of each support 30 is pivotably coupled to collar 26. For example, a hinge (e.g., 82) may be coupled to first end 98 of support 30. In some configurations, hinge 82 may be coupled to first end 98, second end 100, or both to facilitate movement of arm(s) 22. In other configurations, first end 98 or second end 100 may be fixedly, slidably, or pivotably coupled in any suitable fashion, such as, for example, via fasteners, adhesives, and/or the like. For example, first end 98 may be fixed to collar 26 and each support 30 may be flexible such that each support may be deformable from a substantially straight alignment in the closed position to an arcuate alignment in the open position.

In some configurations, second end 100 of support may be coupled to arm (e.g., 22) at a point that is nearer to distal end 76 of the arm than to proximal end 78 of the arm. In other configurations, support(s) 30 may be coupled to arm(s) 22 at a point that is nearer to proximal end 78 of the arm than to distal end 76. As mentioned above, the portion of the arm 22 has a free portion extending from the connection to the second end 100 to the distal end 76. The free portion may be a sufficient length (e.g., approximately 1 to 2 mm) to be able to engage with the foreign object. In some configurations, support 30 may have a uniform thickness or can be tapered at first and second ends 98, 100 to facilitate flexibility of the support. For example, each support 30 is sized and shaped to enable movement of each arm 22 from a closed position to an open position. For example, each support 30 may have a thickness 106 that is greater than or equal to any one of, or between any two of: 0.05, 0.1, 0.15, 0.2, 0.25, 0.3, 0.35, 0.4, 0.45, 0.5, 0.55, 0.6, 0.65, 0.7, 0.75, 0.8, 0.9, 1.0 inches (in.) (e.g., approximately 0.35 in).

One or more components of filter retrieval device 10 (e.g., grapple 14, inner catheter 50, outer catheter 62) may be provided individually and can be combined with each other prior to operation to form the filter retrieval device 10, or the filter retrieval device 10 can be provided as a disassembled kit which is assembled by a patient or a care provider prior to operation. In some configurations, filter retrieval device 10 may be provided in a package (not shown) where the one or more components of the filter retrieval device are sterile and sealed within the package.

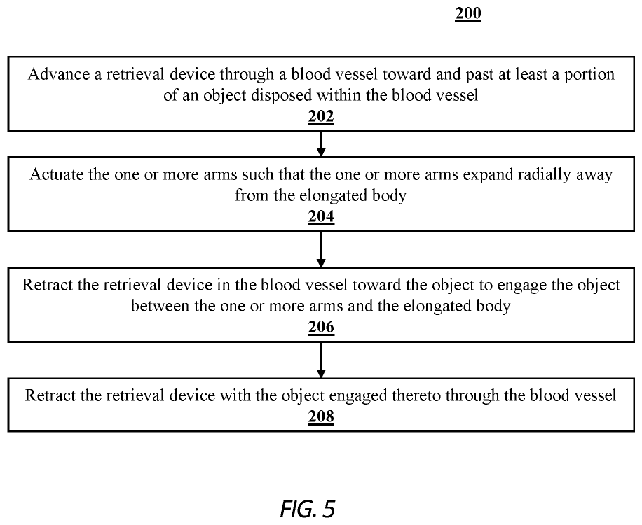

Referring now to FIG. 5, a flowchart of one of the present methods 200 of using a foreign body retrieval device (e.g., 10) is shown. Some of the present methods include a step 202 of advancing a retrieval device (e.g., 10) through a blood vessel toward and past at least a portion of an object (e.g., 108) disposed within the blood vessel. Some of the present methods may include a step 204 of actuating one or more arm(s) (e.g., 22) of the retrieval device such that the one or more arms expand radially away from an elongated body (e.g., 18). Method 200 may include retracting the retrieval device (e.g., 10) in the blood vessel toward the object (e.g., 108) to engage the object between the one or more arms (e.g., 22) and the elongated body (e.g., 18) at step 206. In some such methods, the arm(s) may be actuated such that each arm moves radially toward the elongated body. In some configurations, actuating the arm(s) to move radially toward the elongated body secures the object between the one or more arms and the elongated body. Method 200 may further comprise a step 208 of retracting the retrieval device with the object engaged thereto through the blood vessel.

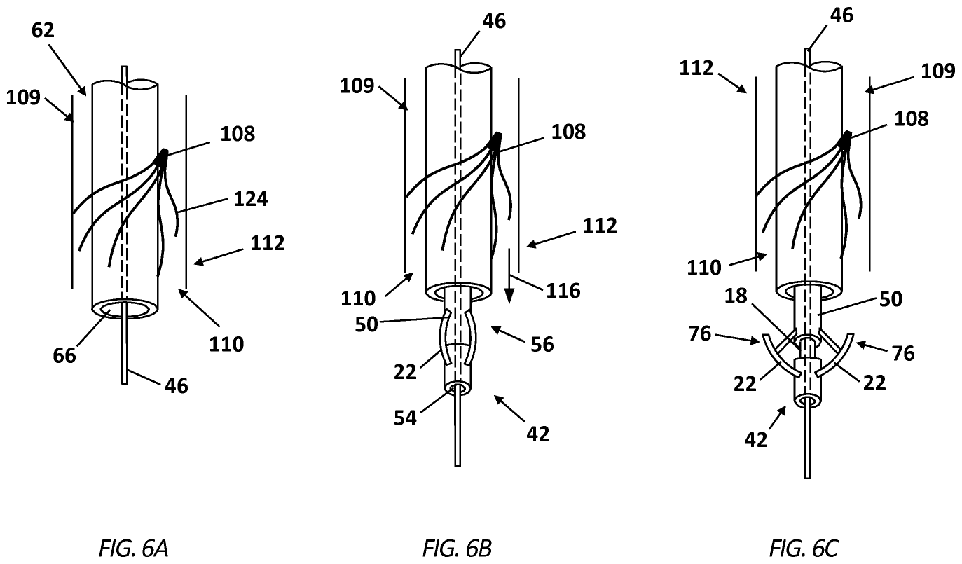

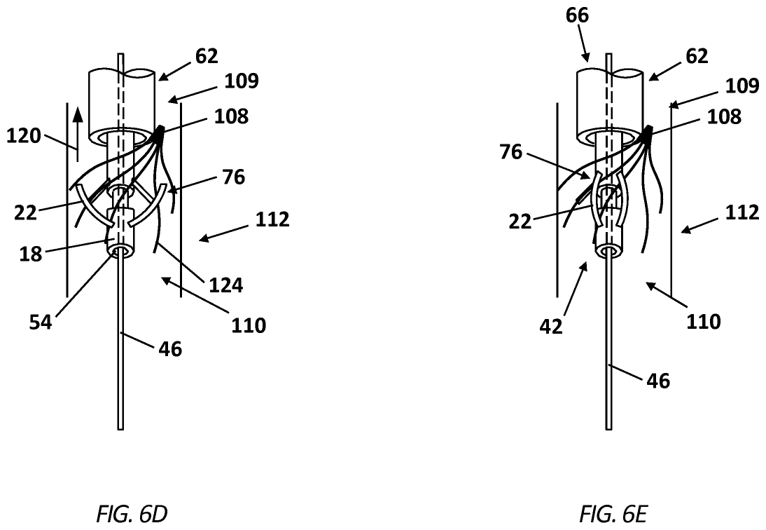

FIGS. 6A-6E depict an example of a method (e.g., 200) of operating a foreign body retrieval device 10, such as a filter retrieval device, to remove an object 108 (e.g. IVC filter) from a patient. The filter retrieval device 10 can comprise any of the above-described filter removal device components (e.g., elongated body 18, arm(s) 22, support(s) 30, inner catheter 50, outer catheter 62, hinge 82, collar 26, guidewire 46, and/or the like).

As shown at least in FIGS. 6A-6B, some of the present methods include retrieving or removing a foreign object 108 (e.g., IVC filter) that is within a blood vessel (e.g., 112) of a patient. In the depicted configuration, foreign object 108 is positioned between a proximate portion 109 and a distal portion 110 of the blood vessel 112. Some of the present methods comprise a step of advancing a filter retrieval device (e.g., 10) (e.g., any of those described above) through the blood vessel 112 of the patient to a foreign object (e.g., 108). To illustrate, filter retrieval device 10 may be advanced so that at least a portion of the filter retrieval device 10 is between proximate portion 109 and distal portion 110 of blood vessel 112. For example, in some methods, distal end 65 of the outer catheter 62 is advanced to the distal portion 110 of the blood vessel 112. In some other methods, distal end 65 of the outer catheter 62 remains at proximal portion 109 of the blood vessel 112. In such methods, a viewing device (e.g., camera) (not shown) may be placed on distal end 65 of outer catheter 62 and an operator may view foreign object 108 during operation. In some methods, a guidewire (e.g., 46) may be used to introduce the grapple (e.g., 14) into the blood vessel 112. For example, grapple 14 may be advanced over the guidewire 46 to a position adjacent to foreign object 108. In the foregoing methods, outer catheter 62 may be flexible to allow for quick and accurate positioning of grapple 14.

Some of the present methods comprise advancing the grapple through a catheter (e.g., 50, 62) disposed within the blood vessel 112. For example, as shown in FIG. 6B, some methods comprise advancing the grapple 14 through an outer catheter (e.g., 62) in a first direction 116 (from a proximate portion 109 of the blood vessel 112 toward a distal portion 110 of the blood vessel). In some such methods, elongated body (e.g., 18) is advanced through lumen (e.g., 66) and past an opening of the distal end 65 of the catheter 62. As shown, in some of the present methods, grapple 14 is advanced out of the distal end 65 and past the foreign object 108 to the distal portion 110 of the blood vessel 112. In some configuration, grapple 14 is advanced out of outer catheter 62 at proximate portion 109 to allow imaging (e.g., via the viewing device) while the foreign object 108 is being engaged and removed.

Referring now to FIG. 6C, to engage foreign object 108 (e.g., IVC filter) disposed within the blood vessel (e.g., 112), some methods include navigating the filter removal device (e.g., 10) past at least a portion of the foreign object 108, optionally to the distal portion 110 of the blood vessel 112, and actuating arm(s) 22 to an open position such that the arm(s) expand radially away from the catheter (e.g., 50, 62) and/or elongated body 18. For example, in some methods actuating arm(s) 22 may comprise opening arm(s) 22 from a closed position (FIG. 6B) to an open position (FIG. 6C). To illustrate, some methods comprise the step of moving distal end 42 of elongated body 18 closer to inner catheter 50 to open arm(s) 22 from the closed position to the open position. For example, in some methods, operator may actuate (e.g., open) arms(s) 22 by moving a portion of filter retrieval device 10 (e.g., 38, 58) that is disposed outside of the blood vessel. Some methods may include the step of using fluoroscopy to view the grapple 14 and/or foreign object 108 when operating the filter retrieval device 10.

Referring now to FIG. 6D, some methods comprise a step of navigating the grapple (e.g., 14) towards foreign object 108 (e.g., IVC filter). In some methods, navigating the grapple comprises a step of advancing grapple 14 in the first direction 116 (FIG. 6B, first direction being from the proximal portion 109 towards the distal portion) past at least a portion of foreign object 108 and then retreating grapple 14 in a second direction 120 (FIG. 6D) opposite the first direction. For example, after actuating arm(s) 22, some methods comprise the step of moving grapple 14 in a second direction 120 toward the proximate portion 109 of the blood vessel 112. In this way, arm(s) 22 may be used to hook foreign object 108 and drag the object toward the second direction 120. Some methods comprise, clamping or locking inner catheter 50 and grapple 14 so that arms 22 remain in a fixed position when the filter retrieval device is navigated through blood vessel 112. Some methods of navigating grapple 14 comprise moving the grapple toward foreign object 108 such that distal ends (e.g., 76) of the arm(s) contact the foreign object. In some steps, the grapple 14 is navigated so that a portion of foreign object 108 is positioned between the arm(s) and the elongated body. For example, in methods where foreign object 108 is an IVC filter, grapple 14 may be navigated so that a strut 124 of IVC filter 108 is interposed between arm(s) 22 and elongated body 18. In this manner, grapple 14 may both clamp and hook foreign object 108 to secure the object so that the object remains engaged with the grapple 14 as the foreign body retrieval device is pulled in second direction 120.

Referring now to FIG. 6E, some methods comprise, actuating the arm(s) 22 to a closed position after navigating grapple toward foreign object 108. For example, some methods comprise closing arm(s) 22 from an open position (FIG. 6D) to a closed position (FIG. 6E). In some methods, closing arm(s) may comprise the steps of moving distal end 42 of elongated body 18 away from inner catheter 50. To secure the foreign object 108, arm(s) 22 are actuated so as to move distal ends 76 of the arm(s) 22 radially towards elongated body 18 to thereby secure the foreign object 108 between the arms 22 and the elongated body 18. The foreign object can be retrieved from the blood vessel by retreating the filter retrieval device 10 with foreign object 108 secured thereto through the blood vessel 112 in the direction 120. For example, some methods include a step of transporting the foreign object through the blood vessel but outside the outer catheter 62. In some methods, the object may be transported within outer catheter 62. For example, some methods comprise the steps of moving grapple 14, inner catheter 50, and foreign object 108 (e.g., IVC filter) within lumen 66 of outer catheter 62. Some methods comprise removing foreign object 108 from blood vessel 112 of the patient.

The above specification and examples provide a complete description of the structure and use of illustrative configurations. Although certain configurations have been described above with a certain degree of particularity, or with reference to one or more individual configurations, those skilled in the art could make numerous alterations to the disclosed configurations without departing from the scope of this invention. As such, the various illustrative configurations of the methods and systems are not intended to be limited to the particular forms disclosed. Rather, they include all modifications and alternatives falling within the scope of the claims, and configurations other than the one shown may include some or all of the features of the depicted configurations. For example, elements may be omitted or combined as a unitary structure, connections may be substituted, or both. Further, where appropriate, aspects of any of the examples described above may be combined with aspects of any of the other examples described to form further examples having comparable or different properties and/or functions, and addressing the same or different problems. Similarly, it will be understood that the benefits and advantages described above may relate to one configuration or may relate to several configurations. Accordingly, no single implementation described herein should be construed as limiting and implementations of the disclosure may be suitably combined without departing from the teachings of the disclosure.

The previous description of the disclosed implementations is provided to enable a person skilled in the art to make or use the disclosed implementations. Various modifications to these implementations will be readily apparent to those skilled in the art, and the principles defined herein may be applied to other implementations without departing from the scope of the disclosure. Thus, the present disclosure is not intended to be limited to the implementations shown herein but is to be accorded the widest scope possible consistent with the principles and novel features as defined by the following claims. The claims are not intended to include, and should not be interpreted to include, means-plus- or step-plus-function limitations, unless such a limitation is explicitly recited in a given claim using the phrase(s) “means for” or “step for,” respectively.

1. A device for retrieving an object within a blood vessel of a patient, the device comprising:
an elongated body configured to be disposed within a catheter, the elongated body having a proximal end, and a distal end opposite the proximal end; and
one or more arms configured to be actuated between a closed position and an open position for engaging a foreign object between the arms and the elongated body, each arm having:
a proximal end coupled to the distal end of the elongated body, and
a distal end extending toward the proximal end of the elongated body in the closed position, the distal end being free;
a collar disposed around the elongated body; and
one or more support members, each support member coupled to the collar and a respective arm of the one or more arms;
wherein:
in the open position, the arm extends radially away from the elongated body to define a first maximum transverse dimension of the device;
in the closed position, the distal end of the arm is closer to the elongated body than when in the open position to define a second maximum transverse dimension of the device that is smaller than the first maximum transverse dimension; and
movement of the collar away from the distal end of the elongated body moves the one or more arms toward the closed position.

2. The device of claim 1,
wherein each support member is configured such that relative movement of the collar relative to the elongated body causes the respective arm to move between the open position and the closed position.

3. The device of claim 1, wherein each support member comprises:
a first end coupled to the collar; and
a second end coupled to the respective arm.

4. The device of claim 3, wherein the first end of each support member is pivotably coupled to the collar such that as the collar moves toward the distal end of the elongated body, the distal end of each of the arms move radially outward away from the elongated body.

5. The device of claim 1, further comprising an inner catheter to which the collar is coupled.

6. The device of claim 1, wherein the elongated body defines a lumen extending through the elongated body between proximal end and distal end.

7. The device of claim 1, wherein the elongated body is movable relative to the collar such that when a distance between the collar and the distal end of the elongated body increases, the distal end of each arm moves towards the proximal end of the elongated body.

8. The device of claim 7, wherein the elongated body is movable relative to the collar such that when the distance between the collar and the distal end of the elongated body increases, the distal end of each arm moves radially towards the elongated body.

9. The device of claim 8, wherein the elongated body is movable relative to the collar such that when the distance between the collar and the distal end of the elongated body decreases, the distal end of each arm moves:
radially away from the elongated body.

10. The device of claim 1, wherein when the first maximum transverse dimension is greater than or equal to 2.667 mm and/or the second maximum transverse dimension is less than or equal to 2.33 mm.

11. The device of claim 5, wherein each of the arms has a maximum transverse dimension that is less than or equal to 0.35 in and wherein an inner diameter of the inner catheter is greater than or equal to the second maximum transverse dimension.

12. The device of claim 3, wherein the second end of the support member is disposed between the first end of the support member and the distal end of the elongated body.

13. The device of claim 3, wherein:
the proximal end of each arm is pivotably coupled to the elongated body; and
the second end of each support member is pivotably coupled to the respective arm.

14. A kit comprising:
the device of claim 1; and
a package wherein the device is sterile and sealed in the package.

15. A method of removing an object within a blood vessel of a patient, the method comprising:
advancing a retrieval device though the blood vessel toward and past at least a portion of an object disposed within the blood vessel, the retrieval device comprising:
an elongated body; and
one or more arms each having a proximal end coupled to the elongated body;
actuating the one or more arms such that the one or more arms expand radially away from the elongated body;
retracting the retrieval device in the blood vessel toward the object;
moving a collar of the retrieval device away from a distal end of a distal end of the elongated body such that the one or more arms contract radially toward the elongated body to engage the object between the one or more arms and the elongated body; and
retracting the retrieval device with the object engaged thereto through the blood vessel.

16. The method of claim 15, wherein:
the one or more arms include a plurality of arms having a first end coupled to the distal end of the elongated body and a second end that is free; and
the plurality of arms are not directly coupled to each other.

17. The method of claim 15, wherein
the retrieval device is advanced through a catheter disposed within the blood vessel; and
the retrieval device is advanced to extend out of a distal end of the catheter to engage the object in the blood vessel.

18. The method of claim 17, wherein
the retrieval device is retracted through the catheter disposed within the blood vessel after engaging the object in the blood vessel.

19. The method of claim 15, wherein a guidewire is used to advance and retract the retrieval device through the blood vessel.

20. The method of claim 15, wherein the retrieval device comprises:
one or more support members, each support member coupled to a respective arm of the one or more arms; and
wherein:
the elongated body comprises a proximal end, and a distal end opposite the proximal end; and
the proximal end of each arm pivotably coupled to the distal end of the elongated body, and
the distal end of each arm extending toward the proximal end of the elongated body.
